(19) United States
(12) Patent Application Publication
(10) Pub. No.: US 2014/0072543 A1
(43) Pub. Date: Mar. 13, 2014

(54) COMPOSITION COMPRISING PROBIOTIC BACTERIA CAPABLE OF RESTORING THE BARRIER EFFECT OF THE STOMACH WHICH IS LOST DURING PHARMACOLOGICAL TREATMENT OF GASTRIC HYPERACIDITY

(71) Applicant: Giovanni Mogna, Novara (NO) (IT)

(72) Inventor: Giovanni Mogna, Novara (NO) (IT)

(21) Appl. No.: 14/113,211

(22) PCT Filed: Apr. 18, 2012
(86) PCT No.: PCT/IB12/00779

§ 371 Date: Nov. 26, 2013

(30) Foreign Application Priority Data
Apr. 20, 2011 (IT) MI2011A000679

(51) Int. Cl. A61K 35/74

(52) U.S. Cl. CPC A61K 35/745; A61K 35/747
USPC 424/93.45; 435/252.1; 435/252.9; 424/93.4

(57) ABSTRACT

A composition comprising probiotic bacteria for use in the pharmacological treatment of gastric hyperacidity is described. The composition is capable of restoring the barrier effect of the stomach which is lost during pharmacological treatment of gastric hyperacidity and minimizes the secondary effects due to said pharmacological treatment.

The present invention relates to a composition comprising probiotic bacteria for use in the pharmacological treatment of gastric hyperacidity. Said composition is capable of restoring the barrier effect of the stomach which is lost during pharmacological treatment of gastric hyperacidity and of minimizing the secondary effects due to said pharmacological treatment.

Over the last decades various pharmacological approaches have been developed for pharmacologically treating gastric hyperacidity, a condition that, if present in an accentuated manner and for a long time, can give rise to various complications or pathologies such as peptic ulcer and gastroesophageal reflux disease.

Among the most widely used drugs there are those based on active ingredients capable of inhibiting histamine H₂ receptor inhibitors such as, for example, cimetidine, famotidine, nizatidine and ranitidine or based on active ingredients capable of inhibiting prostaglandins such as, for example, misoprostol. Another category of drugs is based on active ingredients that perform a function of gastric mucosa protectors such as, for example, bismuth salts, sucralfate or antimuscarinic or parasympatholytic drugs based on pirenzepine and pipenzolate. Finally, there are also antacids such as, for example, sodium bicarbonate, aluminium hydroxide or magnesium hydroxide and proton pump inhibitors based on Lansoprazole, Esomeprazole, Rabeprazole, Pantoprazole and Omeprazole. Proton pump inhibitors (PPI) are a group of molecules whose principal action is represented by a pronounced reduction of the acidity of gastric juices for a fairly long period of time (from 18 to 24 hours).

The group containing PPIs is the successor of the H₂ antihistamines and PPI inhibitors are much more widespread than the latter given their greater effectiveness.

The above-mentioned medicines are used in the symptomatic and etiological treatment of various syndromes, such as (i) dyspepsia; and (ii) gastroduodenal ulcer. PPIs are used to treat or prevent gastric and duodenal ulcers. They are also used in association with several antibiotics in the treatment of gastritis caused by Helicobacter pylori; (iii) Zollinger-Ellison syndrome and (iv) gastroesophageal reflux disease.

PPIs are also used in patients treated with acetyl salicylic acid or other long-term NSAIDs. Such drugs, by inhibiting the function of the enzyme cyclooxygenase 1 (COX 1), have a side effect of reducing prostaglandin synthesis, a process that depends on that very enzyme. Since the functions of prostaglandins include protecting the gastric mucosa from acidity, PPIs are used to reduce acidity and protect the gastric mucosa. This type of medicine inhibits the gastric enzyme H⁺/K⁺-ATPase (proton pump), catalyser of the exchange of H⁺ and K⁺ ions. This creates an effective inhibition of acid secretion.

In the microchannel where the pH is low, close to 2, these inhibitors are ionized and transformed into molecules capable of establishing covalent bonds with the thiol group (SH) of cysteine of the subunit of the pump. Thus the pump is irreversibly inhibited. The resumption of pumping activity entails the production of new pumps, an event which requires 18 to 24 hours on average. A single dose of PPIs therefore enables gastric secretion to be inhibited for about 24 hours. The fact that the inhibitors are active only in an acidic environment explains why they have a minimal effect on the extra-gastric H⁺/K⁺-ATPase situated at the level of the rectum and colon.

In any case, above and beyond their specific mechanism of action, the final effect of nearly all of these classes of drugs for treating gastric hyperacidity or the other pathological conditions mentioned above is to increase the gastric pH, with kinetics and intensities that depend on the specific pharmacological molecule taken and the dosage thereof. Exceptions in this respect are prostaglandins and gastric mucosa protector drugs, which, instead of reducing the intraluminal hydrogen ion concentration, increase the synthesis of mucus and bicarbonate ions by the cells of the gastric wall, thereby increasing the protection of the mucosa against the acidity of the lumen. In any case, the drugs capable of reducing gastric hyperacidity constitute the treatment of choice in the case of peptic ulcers or gastroesophageal reflux, whereas mucosa protectors represent a complementary therapy.

It is well known, though, that a normal gastric acidity constitutes an effective barrier against potentially harmful or pathogenic microorganisms ingested in a normal diet. Many of them, in fact, are particularly sensitive to acidity and are not able to survive for more than 5 minutes, sometimes even less, at pH values lower than 3. It follows that many pathogens, including those belonging to the genus Salmonella, do not reach the intestine alive and, unless there are harmful effects on the human body mediated by secreted toxins already present in the food, they are not capable of giving rise to an intestinal infection and hence a full-blown food intoxication.

It should be said, however, that raising the gastric pH values typically found in subjects who take drugs to reduce or treat gastric hyperacidity renders the same subjects more exposed to risks of food toxinfections caused above all by the consumption of raw foods, especially fish, meat and eggs.

Subjects who take drugs such as, for example, proton pump inhibitors, to reduce or treat gastric hyperacidity, exhibit a stomach pH value of around 5. This pH value allows Enterobacteriaceae and particular strains of E. Coli, endowed with marked decarboxylase activity, to pass intact through the degraded gastric barrier. The proteins ingested in the diet are enzymatically degraded to amino acids, which, in the presence of decarboxylase activity, are modified into a series of biogenic amines such as, for example histamine, tyramine, putrescine and cadaverine, which range from potentially dangerous to very dangerous. The most common symptoms that these biogenic amines can cause are wholly similar to the secondary effects caused by the use of proton pump inhibitors (PPI) and they are: diarrhoea, headache, nausea, abdominal pains and flatulence. Moreover, when certain biogenic amines react with nitrites we have the formation of N-nitrosamines. These nitrosamines provoke a genetic mutation by alkylating DNA and their presence is associated with stomach, intestinal, pancreatic and bladder cancer as well as leukaemia.

Suspending the pharmacological therapy obviously does not represent a possible solution for these subjects, since that would once again expose the gastric or esophageal mucosa to the harmful effects mediated by gastric juices. On the other hand, it is unthinkable to continue the pharmacological therapy and leave the subjects exposed to such risks of infection.

Thus, there remains a necessity to enable subjects in need to take drugs to reduce or treat gastric hyperacidity, on the one hand, and to avoid being exposed to highly dangerous pathogenic infections or to risks of relapsing pathogenic infections, on the other hand.

The Applicant has provided an answer to the above-mentioned necessity thanks to a composition capable of restoring the functionality of the gastric barrier which has a protective effect against pathogenic or harmful microorganisms.

The composition of the present invention is capable of restoring the gastric barrier function which is normally performed by gastric juices and is particularly reduced in subjects who take drugs to reduce or treat gastric hyperacidity. Said composition is capable of minimizing the secondary effects associated with a pharmacological therapy based on proton pump inhibitor drugs (PPIs for short).

The composition of the present invention can be validly used in the treatment of peptic ulcer or gastroesophageal reflux.

After intense research activity, the Applicant surprisingly found that a selected combination of probiotic bacteria is capable of enabling subjects in need to take drugs to reduce or treat gastric hyperacidity, on the one hand, and to avoid being exposed to highly dangerous pathogenic infections or to risks of relapsing pathogenic infections, on the other hand.

Therefore, the subject matter of the present invention is a composition having the features described in the appended independent claim.

Other preferred embodiments of the present invention are described hereunder and will be claimed in the appended dependent claims.

Table 1 shows, by way of example, a group of microorganisms which has valid application in the context of the present invention.

The Applicant conducted an intense activity of research and selection, at the end of which it found that the probiotic bacterial strains belonging to a species selected from the group comprising L. acidophilus, L. crispatus, L. gasseri, L. delbrueckii, L. salivarius, L. casei, L. paracasei, L. plantarum, L. rhamnosus, L. reuteri, L. brevis, L. buchneri, L. fermentum, L. lactis, L. pentosus, B. adolescentis, B. angulatum, B. bifidum, B. breve, B. catenulatum, B. infantis, B. lactis, B. longum, B. pseudocatenulatum and S. thermophilus have valid application in the treatment of subjects who take drugs to reduce or treat gastric hyperacidity.

The bacterial strains were selected because they are capable of colonizing the stomach at a pH value comprised between 4.5 and 5. At this pH value the selected strains act by producing active substances such as bacteriocins and/or metabolites and/or hydrogen peroxide.

The composition of the present invention can be a food composition, for example a symbiotic composition, or else a supplement or a pharmaceutical composition or a medical device.

In a preferred embodiment, the composition can comprise one or more strains selected from among those listed in Table 1.

TABLE 1

| | | Comm. | Depositing | Deposit | Deposit | |
|---|---|---|---|---|---|---|
| No. | Name | code | institution | number | date | Depositor |
| 1 | Lactobacillus casei | LF1i | CNCM I.P. | I-785 | 21 Jul. 1988 | Anidral Srl |
| 2 | Lactobacillus gasseri | LF2i | CNCM I.P. | I-786 | 21 Jul. 1988 | Anidral Srl |
| 3 | Lactobacillus crispatus | LF3i | CNCM I.P. | I-787 | 21 Jul. 1988 | Anidral Srl |
| 4 | Lactobacillus fermentum | LF41 | CNCM I.P. | I-788 | 21 Jul. 1988 | Anidral Srl |
| 5 | Lactobacillus fermentum | LF5 | CNCM I.P. | I-789 | 21 Jul. 1988 | Anidral Srl |
| 6 | Lactobacillus casei ssp. | LFH.i | CNCM I.P. | I-790 | 21 Jul. 1988 | Anidral Srl |
| | pseudopiantarum | | | | | |
| 7 | Streptococcus thermophilus | | BCCM LMG | LMG P-18383 | 5 May 1998 | Anidral Srl |
| | B39 | | | | | |
| 8 | Streptococcus thermophilus | | BCCM LMG | LMG P-18384 | 5 May 1998 | Anidral Srl |
| | T003 | | | | | |
| 9 | Lactobacillus pentosus | | BCCM LMG | LMG P-21019 | 16 Oct. 2001 | Mofin Srl |
| | 9/1 ei | | | | | |
| 10 | Lactobacillus plantarum | LP 02 | BCCM LMG | LMG P-21020 | 16 Oct. 2001 | Mofin Srl |
| | 776/1 bi | | | | | |
| 11 | Lactobacillus plantarum | LP 01 | BCCM LMG | LMG P-21021 | 16 Oct. 2001 | Mofin Srl |
| | 476LL/20 bi | | | | | |
| 12 | Lactobacillus plantarum | | BCCM LMG | LMG P-21022 | 16 Oct. 2001 | Mofin Srl |
| | PR ci | | | | | |
| 13 | Lactobacillus plantarum | | BCCM LMG | LMG P-21023 | 16 Oct. 2001 | Mofin Srl |
| | 776/2 hi | | | | | |
| 14 | Lactobacillus casei ssp. | LPC00 | BCCM LMG | LMG P-21380 | 31 Jan. 2002 | Anidral Srl |
| | paracasei 181A/3 aiai | | | | | |
| 15 | Lactobacillus belonging to | LA 02 | BCCM LMG | LMG P-21381 | 31 Jan. 2002 | Anidral Srl |
| | the acidophilus group | | | | | |
| | 192A/1 aiai | | | | | |
| 16 | Bifidobacterium longum | | BCCM LMG | LMG P-21382 | 31 Jan. 2002 | Anidral Srl |
| | 175A/1 aiai | | | | | |
| 17 | Bifidobacterium breve | | BCCM LMG | LMG P-21383 | 31 Jan. 2002 | Anidral Srl |
| | 195A/1 aici | | | | | |
| 18 | Bifidobacterium lactis | BS 01 | BCCM LMG | LMG P-21384 | 31 Jan. 2002 | Anidral Srl |
| | 32A/3 aiai | | | | | |
| 19 | Lactobacillus plantarum | COAKTIV | BCCM LMG | LMG P-21385 | 31 Jan. 2002 | Mofin Srl |
| | 501/2 gi | | | | | |
| 20 | Lactobacillus lactis ssp. lactis | | BCCM LMG | LMG P-21388 | 31 Jan. 2002 | Mofin Srl |
| | 501/4 ci | | | | | |
| 21 | Lactobacillus lactis ssp. lactis | | BCCM LMG | LMG P-21387 | 15 Mar. 2002 | Mofin Srl |
| | 501/4 hi | | | | | |
| 22 | Lactobacillus lactis ssp. lactis | | BCCM LMG | LMG P-21388 | 31 Jan. 2002 | Mofin Srl |
| | 501/4 ci | | | | | |
| 23 | Lactobacillus plantarum | | BCCM LMG | LMG P-21389 | 15 Mar. 2002 | Mofin Srl |
| | 501/4 li | | | | | |
| 24 | Lactobacillus acidophilus | LA08 | BCCM LMG | LMG P-26144 | 03 Nov. 2010 | Probiotical SpA |
| 25 | Lactobacillus paracasei ssp | LPC10 | BCCM LMG | LMG P-26143 | 03 Nov. 2010 | Probiotical SpA |
| | paracasei | | | | | |
| 26 | Streptococcus thermophilus | GB1 | DSMZ | DSM 16506 | 18 Jun. 2004 | Anidral Srl |
| 27 | Streptococcus thermophilus | GB5 | DSMZ | DSM 16507 | 18 Jun. 2004 | Anidral Srl |
| 28 | Streptococcus thermophilus | Y02 | DSMZ | DSM 16590 | 20 Jul. 2004 | Anidral Srl |
| 29 | Streptococcus thermophilus | Y03 | DSMZ | DSM 16591 | 20 Jul. 2004 | Anidral Srl |
| 30 | Streptococcus thermophilus | Y04 | DSMZ | DSM 16592 | 20 Jul. 2004 | Anidral Srl |
| 31 | Streptococcus thermophilus | YO5 | DSMZ | DSM 16593 | 20 Jul. 2004 | Anidral Srl |
| 32 = | Bifidobacterium adolescentis | BA 03 | DSMZ | DSM 16594 | 21 Jul. 2004 | Anidral Srl |
| 56 | | | | | | |
| 33 | Bifidobacterium adolescentis | BA 04 | DSMZ | DSM 16595 | 21 Jul. 2004 | Anidral Srl |
| 34 | Bifidobacterium breve | BR 04 | DSMZ | DSM 16596 | 21 Jul. 2004 | Anidral Srl |
| 35 | Bifidobacterium pseudocatenulatum | BP 01 | DSMZ | DSM 16597 | 21 Jul. 2004 | Anidral Srl |
| 36 | Bifidobacterium pseudocatenulatum | BP 02 | DSMZ | DSM 16598 | 21 Jul. 2004 | Anidral Srl |
| 37 | Bifidobacterium longum | BL 03 | DSMZ | DSM 16603 | 20 Jul. 2004 | Anidral Srl |
| 38 | Bifidobacterium breve | BR 03 | DSMZ | DSM 16604 | 20 Jul. 2004 | Anidral Srl |
| 39 | Lactobacillus casei ssp. | LR 04 | DSMZ | DSM 16605 | 20 Jul. 2004 | Anidral Srl |
| | rhamnosus | | | | | |
| 40 | Lactobacillus delbrueckii | LDB 01 | DSMZ | DSM 16606 | 20 Jul. 2004 | Anidral Srl |
| | ssp. bulgaricus | | | | | |
| 41 | Lactobacillus delbrueckii | LDB 02 | DSMZ | DSM 16607 | 20 Jul. 2004 | Anidral Srl |
| | ssp. bulgaricus | | | | | |
| 42 | Staphylococcus xylosus | SX 01 | DSMZ | DSM 17102 | 01 Feb. 2005 | Anidral Srl |
| 43 = | Bifidobacterium adolescentis | BA 02 | DSMZ | DSM 17103 | 01 Feb. 2005 | Anidral Srl |
| 57 | | | | | | |
| 44 | Lactobacillus plantarum | LP 07 | DSMZ | DSM 17104 | 01 Feb. 2005 | Anidral Srl |
| 45 | Streptococcus thermophilus | YO8 | DSMZ | DSM 17843 | 21 Dec. 2005 | Anidral Srl |
| 46 | Streptococcus thermophilus | YO9 | DSMZ | DSM 17844 | 21 Dec. 2005 | Anidral Srl |
| 47 | Streptococcus thermophilus | YO100 | DSMZ | DSM 17845 | 21 Dec. 2005 | Anidral Srl |
| 48 | Lactobacillus fermentum | LF06 | DSMZ | DSM 18295 | 24 May 2006 | Anidral Srl |
| 49 | Lactobacillus fermentum | LF07 | DSMZ | DSM 18296 | 24 May 2006 | Anidral Srl |
| 50 | Lactobacillus fermentum | LF08 | DSMZ | DSM 18297 | 24 May 2006 | Anidral Srl |
| 51 | Lactobacillus fermentum | LF09 | DSMZ | DSM 18298 | 24 May 2006 | Anidral Srl |
| 52 | Lactobacillus gasseri | LGS01 | DSMZ | DSM 18299 | 24 May 2006 | Anidral Srl |
| 53 | Lactobacillus gasseri | LGS02 | DSMZ | DSM 18300 | 24 May 2006 | Anidral Srl |
| 54 | Lactobacillus gasseri | LGS03 | DSMZ | DSM 18301 | 24 May 2006 | Anidral Srl |
| 55 | Lactobacillus gasseri | LGS04 | DSMZ | DSM 18302 | 24 May 2006 | Anidral Srl |
| 56 = | Bifidobacterium adolescentis | BA 03 | DSMZ | DSM 18350 | 15 Jun. 2006 | Anidral Srl |
| | EI-3 | | | | | |
| 32 | Bifidobacterium catenulatum | | | | | |
| | sp./pseudocatenulatum EI- | | | | | |
| | 3I, ID 09-255 | | | | | |
| 57 = | Bifidobacterium adolescentis | BA 02 | DSMZ | DSM 18351 | 15 Jun. 2006 | Anidral Srl |
| 43 | EI-15 | | | | | |
| 58 | Bifidobacterium adolescentis | BA 05 | DSMZ | DSM 18352 | 15 Jun. 2006 | Anidral Srl |
| | EI-18 | | | | | |
| | Bifidobacterium animalis | | | | | |
| | subsp. lactis EI-18, ID 09- | | | | | |
| | 256 | | | | | |
| 59 | Bifidobacterium catenulatum | BC 01 | DSMZ | DSM 18353 | 15 Jun. 2006 | Anidral Srl |
| | EI-20 | | | | | |
| 60 | Streptococcus thermophilus | MO1 | DSMZ | DSM 18613 | 13 Sep. 2006 | Mofin Srl |
| | FRai | | | | | |
| 61 | Streptococcus thermophilus | MO2 | DSMZ | DSM 18614 | 13 Sep. 2006 | Mofin Srl |
| | LB2bi | | | | | |
| 62 | Streptococcus thermophilus | MO3 | DSMZ | DSM 18615 | 13 Sep. 2006 | Mofin Srl |
| | LRci | | | | | |
| 63 | Streptococcus thermophilus | MO4 | DSMZ | DSM 18616 | 13 Sep. 2006 | Mofin Srl |
| | FP4 | | | | | |
| 64 | Streptococcus thermophilus | MO5 | DSMZ | DSM 18617 | 13 Sep. 2006 | Mofin Srl |
| | ZZ5F8 | | | | | |
| 65 | Streptococcus thermophilus | MO6 | DSMZ | DSM 18618 | 13 Sep. 2006 | Mofin Srl |
| | TEO4 | | | | | |
| 66 | Streptococcus thermophilus | MO7 | DSMZ | DSM 18619 | 13 Sep. 2006 | Mofin Srl |
| | S1ci | | | | | |
| 67 | Streptococcus thermophilus | MO8 | DSMZ | DSM 18620 | 13 Sep. 2006 | Mofin Srl |
| | 641bi | | | | | |
| 68 | Streptococcus thermophilus | MO9 | DSMZ | DSM 18621 | 13 Sep. 2006 | Mofin Srl |
| | 277A/1ai | | | | | |
| 69 | Streptococcus thermophilus | MO10 | DSMZ | DSM 18622 | 13 Sep. 2006 | Mofin Srl |
| | 277A/2ai | | | | | |
| 70 | Streptococcus thermophilus | MO11 | DSMZ | DSM 18623 | 13 Sep. 2006 | Mofin Srl |
| | IDC11 | | | | | |
| 71 | Streptococcus thermophilus | MO14 | DSMZ | DSM 18624 | 13 Sep. 2006 | Mofin Srl |
| | ML3di | | | | | |
| 72 | Streptococcus thermophilus | MO15 | DSMZ | DSM 18625 | 13 Sep. 2006 | Mofin Srl |
| | TEO3 | | | | | |
| 73 | Streptococcus thermophilus | GG1 | DSMZ | DSM 19057 | 21 Feb. 2007 | Mofin Srl |
| | G62 | | | | | |
| 74 | Streptococcus thermophilus | GG2 | DSMZ | DSM 19058 | 21 Feb. 2007 | Mofin Srl |
| | G1192 | | | | | |
| 75 | Streptococcus thermophilus | GG3 | DSMZ | DSM 19059 | 21 Feb. 2007 | Mofin Srl |
| | GB18 | MO2 | | | | |
| 76 | Streptococcus thermophilus | GG4 | DSMZ | DSM 19060 | 21 Feb. 2007 | Mofin Srl |
| | CCR21 | | | | | |
| 77 | Streptococcus thermophilus | GG5 | DSMZ | DSM 19061 | 21 Feb. 2007 | Mofin Srl |
| | G92 | | | | | |
| 78 | Streptococcus thermophilus | GG6 | DSMZ | DSM 19062 | 21 Feb. 2007 | Mofin Srl |
| | G69 | | | | | |
| 79 | Streptococcus thermophilus | YO 10 | DSMZ | DSM 19063 | 21 Feb. 2007 | Anidral Srl |
| 80 | Streptococcus thermophilus | YO 11 | DSMZ | DSM 19064 | 21 Feb. 2007 | Anidral Srl |
| 81 | Streptococcus thermophilus | YO 12 | DSMZ | DSM 19065 | 21 Feb. 2007 | Anidral Srl |
| 82 | Streptococcus thermophilus | YO 13 | DSMZ | DSM 19066 | 21 Feb. 2007 | Anidral Srl |
| 83 | Weissella ssp. | EX | DSMZ | DSM 19067 | 21 Feb. 2007 | Anidral Srl |
| | WSP 01 | | | | | |
| 84 | Weissella ssp. | EX | DSMZ | DSM 19068 | 21 Feb. 2007 | Anidral Srl |
| | WSP 02 | | | | | |
| 85 | Lactobacillus ssp. | EX | DSMZ | DSM 19069 | 21 Feb. 2007 | Anidral Srl |
| | WSP 03 | | | | | |
| 86 | Lactobacillus plantarum | OY | DSMZ | DSM 19070 | 21 Feb. 2007 | Anidral Srl |
| | LP 09 | | | | | |
| 87 | Lactobacillus plantarum | OY | DSMZ | DSM 19071 | 21 Feb. 2007 | Anidral Srl |
| | LP 10 | | | | | |
| 88 | Lactobacillus lactis | NS 01 | DSMZ | DSM 19072 | 21 Feb. 2007 | Anidral Srl |
| 89 | Lactobacillus fermentum | LF 10 | DSMZ | DSM 19187 | 20 Mar. 2007 | Anidral Srl |
| 90 | Lactobacillus fermentum | LF 11 1251 | DSMZ | DSM 19188 | 20 Mar. 2007 | Anidral Srl |
| 127 | Lactobacillus crispatus | CRL 1266 | DSMZ | DSM 24439 | 04 Jan. 2011 | Probiotical SpA |
| 128 | Lactobacillus paracasei | CRL 1289 | DSMZ | DSM 24440 | 04 Jan. 2011 | Probiotical SpA |
| 129 | Lactobacillus salivarius | CRL 1328 | DSMZ | DSM 24441 | 04 Jan. 2011 | Probiotical SpA |
| 130 | Lactobacillus gasseri | CRL 1259 | DSMZ | DSM 24512 | 25 Jan. 2011 | Probiotical SpA |
| 131 | Lactobacillus acidophilus | CRL 1294 | DSMZ | DSM 24513 | 25 Jan. 2011 | Probiotical SpA |
| 132 | Lactobacillus salivarius | LS04 | DSMZ | DSM 24618 | 02 Mar. 2011 | Probiotical SpA |
| 133 | Lactobacillus crispatus | LCR01 | DSMZ | DSM 24619 | 02 Mar. 2011 | Probiotical SpA |
| 134 | Lactobacillus crispatus | LCR02 | DSMZ | DSM 24620 | 02 Mar. 2011 | Probiotical SpA |
| 135 | Lactobacillus acidophilus | LA09 | DSMZ | DSM 24621 | 02 Mar. 2011 | Probiotical SpA |
| 136 | Lactobacillus gasseri | LGS05 | DSMZ | DSM 24622 | 02 Mar. 2011 | Probiotical SpA |
| 137 | Lactobacillus paracasei | LPC11 | DSMZ | DSM 24623 | 02 Mar. 2011 | Probiotical SpA |
| 138 | Bifidobacterium infantis | BI 02 | DSMZ | DSM 24687 | 29 Mar. 2011 | Probiotical SpA |
| 139 | Bifidobacterium bifidum | BB 06 | DSMZ | DSM 24688 | 29 Mar. 2011 | Probiotical SpA |
| 140 | Bifidobacterium longum | BL 06 | DSMZ | DSM 24689 | 29 Mar. 2011 | Probiotical SpA |
| 141 | Bifidobacterium lactis | BS 07 | DSMZ | DSM 24690 | 29 Mar. 2011 | Probiotical SpA |
| 142 | Bifidobacterium longum | PCB133 | DSMZ | DSM 24691 | 29 Mar. 2011 | Probiotical SpA |
| 143 | Bifidobacterium breve | B632 | DSMZ | DSM 24706 | 07 Apr. 2011 | Probiotical SpA |
| 144 | Bifidobacterium breve | B2274 | DSMZ | DSM 24707 | 07 Apr. 2011 | Probiotical SpA |
| 145 | Bifidobacterium breve | B7840 | DSMZ | DSM 24708 | 07 Apr. 2011 | Probiotical SpA |
| 146 | Bifidobacterium longum | B1975 | DSMZ | DSM 24709 | 07 Apr. 2011 | Probiotical SpA |
| 147 | Lactobacillus salivarius | DLV1 | DSMZ | DSM 25138 | 02 Sep. 2011 | Probiotical SpA |
| 148 | Lactobacillus reuteri | LRE05 | DSMZ | DSM 23139 | 02 Sep. 2011 | Probiotical SpA |
| 149 | Lactobacillus reuteri | LRE06 | DSMZ | DSM 25140 | 02 Sep. 2011 | Probiotical SpA |
| 150 | Lactobacillus reuteri | RC 14 | DSMZ | DSM 25141 | 02 Sep. 2011 | Probiotical SpA |
| 151 | Streptococcus thermophilus | ST 10 | DSMZ | DSM 25246 | 19 Sep. 2011 | Probiotical SpA |
| 152 | Streptococcus thermophilus | ST 11 | DSMZ | DSM 25247 | 19 Sep. 2011 | Probiotical SpA |
| 153 | Streptococcus thermophilus | ST 12 | DSMZ | DSM 25282 | 20 Oct. 2011 | Probiotical SpA |
| 154 | Lactobacillus salivarius | DLV8 | DSMZ | DSM 25545 | 12 Jan. 2012 | Probiotical SpA |
| 155 | Bifidobacterium longum | DLBL 07 | DSMZ | DSM 25669 | 16 Feb. 2012 | Probiotical SpA |
| 156 | Bifidobacterium longum | DLBL 08 | DSMZ | DSM 25670 | 16 Feb. 2012 | Probiotical SpA |
| 157 | Bifidobacterium longum | DLBL 09 | DSMZ | DSM 25671 | 16 Feb. 2012 | Probiotical SpA |
| 158 | Bifidobacterium longum | DLBL 10 | DSMZ | DSM 25672 | 16 Feb. 2012 | Probiotical SpA |
| 159 | Bifidobacterium longum | DLHL 11 | DSMZ | DSM 25673 | 16 Feb. 2012 | Probiotical SpA |
| 160 | Bifidobacterium longum | DLBL 12 | DSMZ | DSM 25674 | 16 Feb. 2012 | Probiotical SpA |
| 161 | Bifidobacterium longum | DLBL 13 | DSMZ | DSM 25675 | 16 Feb. 2012 | Probiotical SpA |
| 162 | Bifidobacterium longum | DLBL 14 | DSMZ | DSM 25676 | 16 Feb. 2012 | Probiotical SpA |
| 163 | Bifidobacterium longum | DLBL 15 | DSMZ | DSM 25677 | 16 Feb. 2012 | Probiotical SpA |
| 164 | Bifidobacterium longum | DLBL 16 | DSMZ | DSM 25678 | 16 Feb. 2012 | Probiotical SpA |
| 165 | Bifidobacterium longum | DLBL 17 | DSMZ | DSM 25679 | 16 Feb. 2012 | Probiotical SpA |
| 166 | Lactobacillus johnsonii | DLLJO 01 | DSMZ | DSM 25680 | 16 Feb. 2012 | Probiotical SpA |
| 167 | Lactobacillus rhamnosus | DLLR 07 | DSMZ | DSM 25681 | 16 Feb. 2012 | Probiotical SpA |
| 168 | Lactobacillus rhamnosus | DLLR 08 | DSMZ | DSM 25682 | 16 Feb. 2012 | Probiotical SpA |
| 169 | Lactobacillus reuteri | DLLRE 07 | DSMZ | DSM 25683 | 16 Feb. 2012 | Probiotical SpA |
| 170 | Lactobacillus reuteri | DLLRE 08 | DSMZ | DSM 25684 | 16 Feb. 2012 | Probiotical SpA |
| 171 | Lactobacillus reuteri | DLLRE 09 | DSMZ | DSM 25685 | 16 Feb. 2012 | Probiotical SpA |
| 172 | Bifidobacterium longum | DLBL 18 | DSMZ | DSM 25708 | 24 Feb. 2012 | Probiotical SpA |
| 173 | Bifidobacterium infantis | BI 03 | DSMZ | DSM 25709 | 24 Feb. 2012 | Probiotical SpA |
| 174 | Lactobacillus plantarum | LP 09 | DSMZ | DSM 25710 | 24 Feb. 2012 | Probiotical SpA |
| 175 | Bifidobacterium longum | DLBL 19 | DSMZ | DSM 25717 | 01 Mar. 2012 | Probiotical SpA |
| 176 | Bifidobacterium longum | DLBL 20 | DSMZ | DSM 25718 | 01 Mar. 2012 | Probiotical SpA |

Preferably, the composition comprises from one to six strains, even more preferably from two to five strains among those listed in Table 1.

Particularly preferred strains are selected from among those listed in Table 2.

TABLE 2

| | Deposit | Antagonized | Strain |
|---|---|---|---|
| Strain | Number | pathogen | properties |
| Lactobacillus pentosus | DSM 21980 | Escherichia coli, | Probiotical |
| LPS 01 | | coliformi | S.p.A. |
| Lactobacillus plantarum | LMG | Escherichia coli, | Probiotical |
| LP 01 | P-21021 | Listeria | S.p.A. |
| | | monocytogenes | |
| Lactobacillus plantarum | LMG | Escherichia coli, | Probiotical |
| LP 02 | P-21020 | Listeria | S.p.A. |
| | | monocytogenes | |
| Lactobacillus plantarum | LMG | Escherichia coli, | Probiotical |
| LP 03 (See Table 1) | P-21022 | Listeria | S.p.A. |
| | | monocytogenes | |
| Lactobacillus plantarum | LMG | Escherichia coli, | Probiotical |
| LP 04 (See Table 1) | P-21023 | Listeria | S.p.A. |
| | | monocytogenes | |
| Lactobacillus pentosus | DSM 21980 | Producer of | Probiotical |
| LPS 01 | | bacteriocins and | S.p.A. |
| | | hydrogen peroxide | |
| Lactobacillus fermentum | CNCM I- | Candida albicans, | Probiotical |
| LF 5 | 789 | Candida krusei, | S.p.A. |
| | | Candida glabrata, | |
| | | Candida parapsilosis | |
| Lactobacillus fermentum | DSM 19187 | Candida albicans, | Probiotical |
| LF 10 | | Candida krusei, | S.p.A. |
| | | Candida glabrata, | |
| | | Candida | |
| | | parapsilosis, | |
| | | Salmonella, | |
| | | Staphylococcus | |
| | | aureus | |
| Lactobacillus fermentum | DSM 18298 | Candida albicans | Probiotical |
| LF 09 | | | S.p.A. |
| Lactobacillus fermentum | DSM 19188 | Candida albicans, | Probiotical |
| LF 11 | | Candida krusei, | S.p.A. |
| | | Candida glabrata, | |
| | | Candida parasilosis | |
| Lactobacillus lactis | DSM 19072 | Bacillus brevis, Bacillus | Probiotical |
| NS 01 | | cereus, Bacillus | S.p.A. |
| | | coagulans, | |
| | | Enterococcus faecalis & | |
| | | faecium, Staphylococcus | |
| | | aureus, Clostridium | |
| | | botulinum, Clostridium | |
| | | butyricum, Listeria | |
| Lactobacillus salivarius | DSM 24618 | Candida, | Probiotical |
| LS04 | | Enterococcus | S.p.A. |
| | | faecalis & faecium, | |
| | | Neisseria | |
| | | gonorrhoeae | |
| Lactobacillus crispatus | DSM 24619 | Strong producer of | Probiotical |
| LCR01 | | hydrogen peroxide/ | S.p.A. |
| | | non-specific, broad- | |
| | | spectrum inhibition | |
| Lactobacillus crispatus | DSM 24620 | Strong producer of | Probiotical |
| LCR02 | | hydrogen peroxide/ | S.p.A. |
| | | non-specific, broad- | |
| | | spectrum inhibition | |
| Lactobacillus acidophilus | DSM 24621 | Candida by | Probiotical |
| LA09 | | coaggregation | S.p.A. |
| Lactobacillus gasseri | DSM 24622 | Strong producer of | Probiotical |
| LGS05 | | lactic acid/ | S.p.A. |
| | | non-specific, broad- | |
| | | spectrum inhibition | |
| Lactobacillus paracasei | DSM 24623 | Staphylococcus | Probiotical |
| LPC11 | | aureus | S.p.A. |
| | | Strong producer of | |
| | | hydrogen peroxide/ | |
| | | non-specific, broad- | |
| | | spectrum inhibition | |
| Lactobacillus rhamnosus | DSM 21981 | Candida krusei, | Probiotical |
| LR 06 | | Candida albicans, | S.p.A. |
| | | Candida glabrata, | |
| | | Escherichia coli, | |
| | | Gardnerella | |
| | | vaginalis | |
| Lactobacillus reuteri | DSM 17938 | Escherichia coli, | BioGaia |
| Lactobacillus reuteri | PTA ATCC 6475 | other colifoms | BioGaia |
| Lactobacillus reuteri | DSM 23877 | Helicobacter pylori, | Probiotical |
| LRE 01 | | Listeria | S.p.A. |
| Lactobacillus reuteri | DSM 23878 | monocytogenes, | Probiotical |
| LRE 02 | | Salmonella | S.p.A. |
| Lactobacillus reuteri | DSM 23879 | typhimurium, | Probiotical |
| LRE 03 | | Pseudomona | S.p.A. |
| Lactobacillus reuteri | DSM 23880 | aeruginosa, Shigella | Probiotical |
| LR 04 | | spp. Campylobacter | S.p.A. |
| | | jejuni, Bacillus | |
| | | subtilis, Clostridium | |
| | | perfringens, Candida | |
| | | albicans, Aspergillus | |
| | | flavus, Tripanosoma | |
| | | cruzi, Eimeria tenella | |
| Lactobacillus reuteri | ATCC 5730 | | BIOGAIA |
| Lactobacillus delbrueckii | DSM 22106 | Klebsiella oxytoca, | Probiotical |
| ssp. delbrueckii DSMZ | | Enterobacter | S.p.A. |
| 20074 | | cloacae, Klebsiella | |
| | | pneumoniae | |
| | | Escherichia coli | |
| Bifidobacterium longum | DSM 24691 | Campylobacter jejuni | Probiotical |
| PCB 133 | | | S.p.A. |
| Bifidobacterium longum | DSM 24689 | Campylobacter jejuni | Probiotical |
| BL06 | | | S.p.A. |
| Bifidobacterium longum | DSM 24709 | Klebsiella oxytoca, | Probiotical |
| B1975 | | Enterobacter | S.p.A. |
| Bifidobacterium breve | DSM 24707 | cloacae, Klebsiella | Probiotical |
| B2274 | | pneumoniae, | S.p.A. |
| Bifidobacterium breve | DSM 24706 | Escherichia coli | Probiotical |
| B632 | | | S.p.A. |
| Bifidobacterium breve | DSM 24708 | | Probiotical |
| B7840 | | | S.p.A. |

The strains in Table 2 were individually tested in order to identify the pathogen they are capable of antagonizing (by inhibiting growth or reducing the number of one or more harmful or pathogenic microbial species/genera), as shown in column 3 of Table 2. Table 2 shows that the bacteria are capable of producing hydrogen peroxide or at least one bacteriocin with an inhibiting activity on one or more harmful or pathogenic microbial species/genera.

All of the strains described and/or claimed in the present patent application have been deposited in accordance with the Budapest Treaty and are made available to the public, on request, by the competent Depositing Authority.

The compositions of the present invention have valid application for use both in the treatment of subjects who take drugs to reduce or treat gastric hyperacidity and in the treatment of an ulcer caused by a deficiency in the protective mechanisms of the mucosa (e.g. reduced secretion or responsiveness to prostaglandin E, as in the case of the intake of aspirin or other NSAIDs) or an H. pylori infection. In other words, the composition of the present invention has application also in subjects whom are prescribed PPIs/other antacid drugs though they do not have gastric hyperacidity, but rather a lesion of the gastric and/or duodenal mucosa resulting from an altered ratio between gastric acidity and the protector mechanisms of the mucosa.

In a preferred embodiment, the composition comprises from one to six strains, preferably from two to five strains, even more preferably three or four, selected from the group comprising or, alternatively, consisting of:

1. Lactobacillus pentosus LPS01 DSM 21980

2. Lactobacillus plantarum LP01 LMG P-21021

3. Lactobacillus plantarum LP02 LMG P-21020

4. Lactobacillus plantarum LP03 LMG P-21022

5. Lactobacillus plantarum LP04 LMG P-21023

6. Lactobacillus rhamnosus LR06 DSM 21981

7. Lactobacillus delbrueckii LDD 01 (MB386) DSM 20074

8. Bifidobacterium longum B1975 DSM 24709

9. Bifidobacterium breve B2274 DSM 24707

10. Bifidobacterium breve B632 DSM 24706

11. Bifidobacterium breve B7840 DSM 24708

12. Bifidobacterium longum PCB 133 DSM 24691

13. Bifidobacterium longum BL06 DSM 24689

In another preferred embodiment, the composition of the present invention comprises from one to six strains, preferably from two to five strains, even more preferably three or four, selected from those indicated above as 1 to 13, in association with the strain Lactobacillus fermentum LF 09 DSM 18298 and/or the strain Lactococcus lactis NS 01 DSM 19072.

In another preferred embodiment, the composition of the present invention comprises from one to six strains, preferably from two to five strains, even more preferably three or four, selected from those indicated above as 1 to 13, in association with at least one strain selected from the group comprising or, alternatively, consisting of:

a. Lactobacillus reuteri LRE 01 DSM 23877

b. Lactobacillus reuteri LRE 02 DSM 23878

c. Lactobacillus reuteri LRE 03 DSM 23879

d. Lactobacillus reuteri LRE 04 DSM 23880

In the composition of the present invention, the mixture of bacterial strains is present in an amount comprised from 0.5 to 20% by weight, relative to the total weight of the composition, preferably from 2.5 to 8%.

In a preferred embodiment, the composition can further comprise at least one prebiotic fibre and/or carbohydrates with bifidogenic action.

The prebiotic fibre that has application in the composition of the present invention is a fibre that must be used by the bacterial strains present in the composition, but not by the pathogens they are intended to antagonize.

If the pathogen to be antagonized belongs to the genus Candida, fructo-oligosaccharides (FOS) and galacto-oligosaccharides (GOS) have valid application, since said fibres are not utilized by Candida. Whereas, gluco-oligosaccharides (GOSα) are capable of directly inhibiting E. coli by means of several metabolites. Therefore, the prebiotic fibre can be selected, depending on circumstances and the pathogen to be antagonized, from among: inulin, fructo-oligosaccharides (FOS), galacto- and trans-galacto-oligosaccharides (GOS and TOS), gluco-oligosaccharides (GOSα), xylo-oligosaccharides (XOS), chitosan oligosaccharides (COS), soy oligosaccharides (SOS), isomalto-oligosaccharides (IMOS), resistant starch, pectin, psyllium, arabinogalactans, glucomannans, galactomannans, xylans, lactosucrose, lactulose, lactitol and various other types of gums, acacia, carob, oat or bamboo fibre, citrus fibres and, in general, fibres containing a soluble and an insoluble portion, in a variable ratio to each other.

In a preferred embodiment of the invention, the composition comprises at least one prebiotic fibre selected from among the ones mentioned above and/or suitable mixtures thereof in any relative percentage. The amount of the prebiotic fibres and/or carbohydrates having a bifidogenic action, if present in the composition, is comprised from 0 to 60% by weight, preferably from 5 to 45% and even more preferably from to 30%, relative to the total weight of the composition. In this case the composition or supplement has symbiotic activity and functional properties. Moreover, the composition can further comprise other active ingredients and/or components, such as vitamins, minerals, bioactive peptides, substances having antioxidant, hypocholesterolemizing, hypoglycemizing, anti-inflammatory or anti-sweetener activity in an amount by weight generally comprised from 0.001% to 20% by weight, preferably from 0.01% to 5%, depending in any case on the type of active component and the recommended daily dose thereof, if defined, relative to the total weight of the composition.

The food composition of the present invention, for example a symbiotic composition, or a supplement or a pharmaceutical composition, is prepared using techniques and equipment known to a person skilled in the art.

In a preferred embodiment, the composition contains bacteria in a concentration comprised from 1×10⁶ to 1×10¹¹ CFU/g of mixture, preferably from 1×10⁸ to 1×10¹⁰ CFU/g of mixture.

In a preferred embodiment, the composition contains bacteria in a concentration comprised from 1×10⁶ to 1×10¹¹ CFU/dose, preferably from 1×10⁸ to 1×10¹⁰ CFU/dose.

The dose can be comprised from 0.2 to 10 g; for example it is 0.25 g, 1 g, 3 g, 5 g or 7 g.

The probiotic bacteria used in the present invention can be in solid form, in particular in powder, dehydrated powder or lyophilized form.

All of the compositions of the present invention are prepared according to techniques known to a person skilled in the art and using known equipment.

In a preferred embodiment, the composition of the present invention further comprises a drug to reduce or treat gastric hyperacidity.

In a preferred embodiment, said drug is selected from the group comprising or, alternatively, consisting of:
H2 receptor inhibitors, preferably cimetidine, famotidine, nizatidine or ranitidine;
prostaglandins, preferably misoprostol;
gastric mucosa protectors, preferably bismuth salts or sucralfate;
muscarinic antagonists or parasympatholytic agents, preferably pirenzepine or pipenzolate;
antacids, preferably sodium bicarbonate, aluminium hydroxide, magnesium hydroxide;
proton pump inhibitors, preferably Lansoprazole, Esomeprazole, Rabeprazole, Pantoprazole and Omeprazole. Preferably, said drug is selected from the group comprising or, alternatively, consisting of:
H2 receptor inhibitors, preferably cimetidine, famotidine, nizatidine or ranitidine;
muscarinic antagonists or parasympatholytic agents, preferably pirenzepine or pipenzolate;
antacids, preferably sodium bicarbonate, aluminium hydroxide, magnesium hydroxide;
proton pump inhibitors, preferably selected from the group comprising Lansoprazole, Esomeprazole, Rabeprazole, Pantoprazole and Omeprazole.

Even more preferably, said drug is selected from the group comprising or, alternatively, consisting of:
H2 receptor inhibitors, preferably cimetidine, famotidine, nizatidine or ranitidine;
proton pump inhibitors, preferably selected from the group comprising Lansoprazole, Esomeprazole, Rabeprazole, Pantoprazole and Omeprazole.

In a preferred embodiment, the composition of the present invention is a pharmaceutical composition comprising the bacteria described in Table 1 or in Table 2 or in the preferred embodiments set forth above, said bacteria being in association with a drug indicated for reducing or treating gastric hyperacidity, as specified above. Advantageously, the drug is a proton pump inhibitor selected from the group comprising Lansoprazole, Esomeprazole, Rabeprazole, Pantoprazole and Omeprazole. Both the bacteria and the drug are intimately present in the same composition. For example, the bacteria and the drug are present together in a tablet, lozenge or granulate in a pharmaceutical form suitable for oral administration. It is essential that the bacteria and the drug be administered simultaneously and act simultaneously, since it is necessary to restore the barrier effect removed by the proton pump inhibitors (PPI) thanks to the action of the probiotic bacteria of the present invention, which produce bacteriocins and are capable of colonizing the stomach thanks to the fact that the proton pump inhibitors have raised the pH to a value of about 4.5-5.0.

In another preferred embodiment, the composition of the present invention is in the form of a medical device. In this case the bacteria are present in a composition suitable for oral administration, such as, for example, a tablet, lozenge or granulate and, separately, the drug indicated for reducing or treating gastric hyperacidity, as specified above, is present in another composition suitable for oral administration. Advantageously, the drug is a proton pump inhibitor selected from the group comprising Lansoprazole, Esomeprazole, Rabeprazole, Pantoprazole and Omeprazole. Therefore, for example, two tablets are administered, one containing the bacteria and the other containing the drug.

In any case, the two tablets must be administered simultaneously, given that it is necessary for the bacteria to act simultaneously with the proton pump inhibitors.

In the case of a medical device as well, it is essential that the bacteria and the drug be administered within a short space of time from each other, since it is necessary to restore the barrier effect removed by the proton pump inhibitors (PPI) thanks to the action of the bacteria, which produce bacteriocins and are capable of colonizing the intestine thanks to the fact that the proton pump inhibitors have raised the pH to a value of about 4.5-5.0.

The Applicant has found that the bacteria selected and listed in Table 1 or Table 2 or in the preferred embodiments mentioned above, are capable of colonizing the stomach at a pH value of around 5 in such a way as to restore the barrier effect reduced or eliminated by the rise in the pH level resulting from the action of the drugs indicated for reducing or treating gastric hyperacidity, such as, for example, a proton pump inhibitor drug selected from the group comprising Lansoprazole, Esomeprazole, Rabeprazole, Pantoprazole and Omeprazole.

In a preferred embodiment, the composition containing the probiotic bacterial strains of the present invention—said strains being capable of producing specific bacteriocins—is also a useful adjuvant to treatments aimed at definitively eliminating Helicobacter pylori and avoiding recurrences thereof.

Therefore, the subject matter of the present invention is a composition comprising at least one strain of bacteria listed in Table 1 or in Table 2 or in one of the embodiments mentioned above, for use in the preventive and/or curative treatment of infections, disorders or diseases caused by the presence of Helicobacter pylori, in particular in the preventive and/or curative treatment of relapses of infections caused by Helicobacter pylori.

In the broadest sense of the term, antibiotics are defined as molecular species which are produced by an organism and are active against the growth of other organisms. In practice, however, antibiotics are generally considered secondary metabolites that are active at low concentrations in blocking the growth of microorganisms. Secondary products of metabolism such as organic acids, ammonia and hydrogen peroxide are not to be included in the category of antibiotics. Antibiotics are molecules, also peptide molecules (penicillin), produced by multi-enzymatic systems whose biosynthesis is not blocked by protein synthesis inhibitors. Bacteriocins, on the other hand, are products of ribosomal synthesis.

Bacteriocins are peptide molecules synthesized by ribosomes which can also be associated with lipids or carbohydrates. Although some bacteriocins produced by Gram-positive bacteria (Lactobacillus, Lactocoocus) possess inhibition spectra limited to some strains belonging to the same species as the producer microorganism, most of them show a broad spectrum of action against various bacterial species, both Gram-positive and Gram-negative. The present classification of bacteriocins is based both on their chemical nature and spectrum of action.

1. A pharmaceutical or food composition or a supplement comprising
at least one bacterial strain belonging to the species: L. acidophilus, L. crispatus, L. gasseri, L. delbrueckii, L. salivarius, L. casei, L. paracasei, L. plantarum, L. rhamnosus, L. reuteri, L. brevis, L. buchneri, L. fermentum, L. lactis, L. pentosus, B. adolescentis, B. angulatum, B. bifidum, B. breve, B. catenulatum, B. infantis, B. lactis, B. longum, B. pseudocatenulatum and S. Thermophilus, which is capable of colonizing the stomach at a pH value comprised from 4.5 to 5 and of producing bacteriocins and/or metabolites and/or hydrogen peroxide,
the pharmaceutical food composition or supplement being in a form suitable for administration of the at least one bacterial strain to the stomach of a subject.

2. The pharmaceutical or food composition or a supplement according to claim 1, wherein said at least one bacterial strain is selected from:
1. Lactobacillus pentosus LPS 01 DSM 21980
2. Lactobacillus plantarum LP 01 LMG P-21021
3. Lactobacillus plantarum LP 02 LMG P-21020
4. Lactobacillus plantarum LP 03 LMG P-21022
5. Lactobacillus plantarum LP 04 LMG P-21023
6. Lactobacillus pentosus LPS 01 DSM 21980
7. Lactobacillus fermentum LF 5 CNCM I-789
8. Lactobacillus fermentum LF 10 DSM 19187
9. Lactobacillus fermentum LF 11 DSM 18298
10. Lactobacillus fermentum LF 11 DSM 19188
11. Lactobacillus lactis NS 01 DSM 19072
12. Lactobacillus salivarius LS04 DSM 24618
13. Lactobacillus crispatus LCR01 DSM 24619
14. Lactobacillus crispatus LCR02 DSM24620
15. Lactobacillus acidophilus LA09 DSM 24621
16. Lactobacillus gasseri LGS05 DSM 24622
17. Lactobacillus paracasei LPC11 DSM 24623
18. Lactobacillus rhamnosus LR 06 DSM 21981
19. Lactobacillus reuteri DSM 17938
20. Lactobacillus reuteri PTA ATCC 6475
21. Lactobacillus reuteri LRE 01 DSM 23877
22. Lactobacillus reuteri LRE 02 DSM 23787
23. Lactobacillus reuteri LRE 03 DSM 23879
24. Lactobacillus reuteri LRE 04 DSM 23880
25. Lactobacillus reuteri ATCC 5730
26. Lactobacillus delbrueckii ssp. Delbrueckii DSMZ 20074
27. Bifidobacterium longum PCB 133 DSM 24691
28. Bifidobacterium longum BL06 DSM 24689
29. Bifidobacterium longum B1975 DSM 24709
30. Bifidobacterium breve B2274 DSM 24707
31. Bifidobacterium breve B632 DSM 24706, and
32. Bifidobacterium breve B7840 DSM 24708.

3. The pharmaceutical or food composition or a supplement use according to claim 1, wherein said composition comprises from one to six strains selected from the group consisting of:
1. Lactobacillus pentosus LPS01 DSM 21980
2. Lactobacillus plantarum LP01 LMG P-21021
3. Lactobacillus plantarum LP02 LMG P-21020
4. Lactobacillus plantarum LP03 LMG P-21022
5. Lactobacillus plantarum LP04 LMG P-21023
6. Lactobacillus rhamnosus LR06 DSM 21981
7. Lactobacillus delbrueckii LDD 01 (MB386) DSM 20074
8. Bifidobacterium longum B1975 DSM 24709
9. Bifidobacterium breve B2274 DSM 24707
10. Bifidobacterium breve B632 DSM 24706
11. Bifidobacterium breve B7840 DSM 24708
12. Bifidobacterium longum PCB 133 DSM 24691
13. Bifidobacterium longum BL06 DSM 24689
in association with the strain Lactobacillus fermentum LF 09 DSM 18298 and/or the strain Lactococcus lactis NS 01 DSM 19072.

4. The pharmaceutical or food composition or a supplement according to claim 3, wherein said composition comprises the one to six strains in association with at least one strain selected from the group consisting of:
a. Lactobacillus reuteri LRE 01 DSM 23877
b. Lactobacillus reuteri LRE 02 DSM 23878
c. Lactobacillus reuteri LRE 03 DSM 23879
d. Lactobacillus reuteri LRE 04 DSM 23880.

5. The pharmaceutical or food composition or a supplement according to claim 1, in a combination with a drug reducing or treating gastric hyperacidity.

6. The pharmaceutical or food composition or a supplement according to claim 5, wherein said drug is selected from the group consisting of:
H2 receptor inhibitors,
muscarinic antagonists, or parasympatholytic agents
antacids, and
proton pump inhibitors.

7. A method to treat a subject taking drugs to reduce or treat gastric hyperacidity, the method comprising administering to the subject an effective amount of the composition according to claim 1.

8. A pharmaceutical composition comprising at least one strain belonging to the species: L. acidophilus, L. crispatus, L. gasseri, L. delbrueckii, L. salivarius, L. casei, L. paracasei, L. plantarum, L. rhamnosus, L. reuteri, L. brevis, L. buchneri, L. fermentum, L. lactis, L. pentosus, B. adolescentis, B. angulatum, B. B. breve, B. catenulatum, B. infantis, B. lactis, B. longum, B. pseudocatenulatum and S. Thermophilus, and a drug suitable to reduce or treat gastric hyperacidity.

9. The composition according to claim 8, wherein said bacterial strains and said drug are present together in a pharmaceutical form for oral use.

10. The method according to claim 7, wherein the composition is administered in an effective amount for preventive and/or curative treatment of infections, disorders or diseases caused by the presence of Helicobacter pylori.

11. The pharmaceutical or food composition or a supplement according to claim 5, wherein said composition comprises at least one bacterial strain selected from
1. Lactobacillus pentosus LPS 01 DSM 21980
2. Lactobacillus plantarum LP 01 LMG P-21021
3. Lactobacillus plantarum LP 02 LMG P-21020
4. Lactobacillus plantarum LP 03 LMG P-21022
5. Lactobacillus plantarum LP 04 LMG P-21023
6. Lactobacillus pentosus LPS 01 DSM 21980
7. Lactobacillus fermentum LF 5 CNCM I-789
8. Lactobacillus fermentum LF 10 DSM 19187
9. Lactobacillus fermentum LF 11 DSM 18298
10. Lactobacillus fermentum LF 11 DSM 19188
11. Lactobacillus lactis NS 01 DSM 19072
12. Lactobacillus salivarius LS04 DSM 24618
13. Lactobacillus crispatus LCR01 DSM 24619
14. Lactobacillus crispatus LCR02 DSM24620
15. Lactobacillus acidophilus LA09 DSM 24621
16. Lactobacillus gasseri LGS05 DSM 24622
17. Lactobacillus paracasei LPC11 DSM 24623
18. Lactobacillus rhamnosus LR 06 DSM 21981
19. Lactobacillus reuteri DSM 17938
20. Lactobacillus reuteri PTA ATCC 6475
21. Lactobacillus reuteri LRE 01 DSM 23877
22. Lactobacillus reuteri LRE 02 DSM 23787
23. Lactobacillus reuteri LRE 03 DSM 23879
24. Lactobacillus reuteri LRE 04 DSM 23880
25. Lactobacillus reuteri ATCC 5730
26. Lactobacillus delbrueckii ssp. Delbrueckii DSMZ 20074
27. Bifidobacterium longum PCB 133 DSM 24691
28. Bifidobacterium longum BL06 DSM 24689
29. Bifidobacterium longum B1975 DSM 24709
30. Bifidobacterium breve B2274 DSM 24707
31. Bifidobacterium breve B632 DSM 24706
32. Bifidobacterium breve B7840 DSM 24708.

12. The pharmaceutical or food composition or a supplement according to claim 5 wherein said drugs are selected from the group consisting of:
an H2 receptor inhibitor, a prostaglandin, a gastric mucosa protector, a muscarinic antagonist or parasympatholytic agents, an antacid, and a proton pump inhibitor.

13. The pharmaceutical or food composition or a supplement according to claim 12, wherein,
the H2 receptor inhibitor is selected from the group consisting of cimetidine, famotidine, nizatidine, and ranitidine,
the prostaglandin is misoprostol,
the gastric mucosa protectors, is selected from the group consisting of bismuth salts, and sucralfate,
the muscarinic antagonists, or parasympatholytic agents, from the group consisting of pirenzepine, or pipenzolate
the antacids is selected from the group consisting of sodium bicarbonate, aluminium hydroxide, and magnesium hydroxide, and
the proton pump inhibitor is selected from the group consisting of Lansoprazole, Esomeprazole, Rabeprazole, Pantoprazole and Omeprazole

14. The pharmaceutical or food composition or a supplement according to claim 6, wherein
the H2 receptor inhibitor is selected from the group consisting of cimetidine, famotidine, nizatidine, and ranitidine,
the muscarinic antagonist or parasympatholytic agents, is pirenzepine, or pipenzolate
the antacids is selected from the group consisting of sodium bicarbonate, aluminium hydroxide, and magnesium hydroxide, and
the proton pump inhibitor is selected from the group consisting of Lansoprazole, Esomeprazole, Rabeprazole, Pantoprazole and Omeprazole

15. The method according to claim 7, wherein said composition comprises at least one bacterial strain selected from
1. Lactobacillus pentosus LPS 01 DSM 21980
2. Lactobacillus plantarum LP 01 LMG P-21021
3. Lactobacillus plantarum LP 02 LMG P-21020
4. Lactobacillus plantarum LP 03 LMG P-21022
5. Lactobacillus plantarum LP 04 LMG P-21023
6. Lactobacillus pentosus LPS 01 DSM 21980
7. Lactobacillus fermentum LF 5 CNCM I-789
8. Lactobacillus fermentum LF 10 DSM 19187
9. Lactobacillus fermentum LF 11 DSM 18298
10. Lactobacillus fermentum LF 11 DSM 19188
11. Lactobacillus lactis NS 01 DSM 19072
12. Lactobacillus salivarius LS04 DSM 24618
13. Lactobacillus crispatus LCR01 DSM 24619
14. Lactobacillus crispatus LCR02 DSM24620
15. Lactobacillus acidophilus LA09 DSM 24621
16. Lactobacillus gasseri LGS05 DSM 24622
17. Lactobacillus paracasei LPC11 DSM 24623
18. Lactobacillus rhamnosus LR 06 DSM 21981
19. Lactobacillus reuteri DSM 17938
20. Lactobacillus reuteri PTA ATCC 6475
21. Lactobacillus reuteri LRE 01 DSM 23877
22. Lactobacillus reuteri LRE 02 DSM 23787
23. Lactobacillus reuteri LRE 03 DSM 23879
24. Lactobacillus reuteri LRE 04 DSM 23880
25. Lactobacillus reuteri ATCC 5730
26. Lactobacillus delbrueckii ssp. Delbrueckii DSMZ 20074
27. Bifidobacterium longum PCB 133 DSM 24691
28. Bifidobacterium longum BL06 DSM 24689
29. Bifidobacterium longum B1975 DSM 24709
30. Bifidobacterium breve B2274 DSM 24707
31. Bifidobacterium breve B632 DSM 24706, and
32. Bifidobacterium breve B7840 DSM 24708.

16. The method of claim 7, wherein gastric hyperacidity is associated to dyspepsia, gastroduodenal ulcer, gastric ulcer, peptic ulcer, duodenal ulcer, gastritis caused by Helicobacter pylori and gastroesophageal reflux disease.

17. The method of claim 7, wherein the composition is in a combination or pharmaceutical composition further comprising the drug suitable to reduce or treat gastric hyperacidity.

18. The pharmaceutical or food composition or a supplement according to claim 8, wherein said composition comprises at least one bacterial strain selected from
1. Lactobacillus pentosus LPS 01 DSM 21980
2. Lactobacillus plantarum LP 01 LMG P-21021
3. Lactobacillus plantarum LP 02 LMG P-21020
4. Lactobacillus plantarum LP 03 LMG P-21022
5. Lactobacillus plantarum LP 04 LMG P-21023
6. Lactobacillus pentosus LPS 01 DSM 21980
7. Lactobacillus fermentum LF 5 CNCM I-789
8. Lactobacillus fermentum LF 10 DSM 19187
9. Lactobacillus fermentum LF 11 DSM 18298
10. Lactobacillus fermentum LF 11 DSM 19188
11. Lactobacillus lactis NS 01 DSM 19072
12. Lactobacillus salivarius LS04 DSM 24618
13. Lactobacillus crispatus LCR01 DSM 24619
14. Lactobacillus crispatus LCR02 DSM24620
15. Lactobacillus acidophilus LA09 DSM 24621
16. Lactobacillus gasseri LGS05 DSM 24622
17. Lactobacillus paracasei LPC11 DSM 24623
18. Lactobacillus rhamnosus LR 06 DSM 21981
19. Lactobacillus reuteri DSM 17938
20. Lactobacillus reuteri PTA ATCC 6475
21. Lactobacillus reuteri LRE 01 DSM 23877
22. Lactobacillus reuteri LRE 02 DSM 23787
23. Lactobacillus reuteri LRE 03 DSM 23879
24. Lactobacillus reuteri LRE 04 DSM 23880
25. Lactobacillus reuteri ATCC 5730
26. Lactobacillus delbrueckii ssp. Delbrueckii DSMZ 20074
27. Bifidobacterium longum PCB 133 DSM 24691
28. Bifidobacterium longum BL06 DSM 24689
29. Bifidobacterium longum B1975 DSM 24709
30. Bifidobacterium breve B2274 DSM 24707
31. Bifidobacterium breve B632 DSM 24706, and
32. Bifidobacterium breve B7840 DSM 24708.

19. The pharmaceutical composition of claim 8, wherein the drug suitable to reduce or treat gastric hyperacidity is selected from the group consisting of H2 receptor inhibitors, prostaglandins, gastric mucosa protectors, muscarinic antagonists, or parasympatholytic agents, antacids, and proton pump inhibitors.

20. The pharmaceutical composition of claim 19, wherein
the H2 receptor inhibitor is selected from the group consisting of cimetidine, famotidine, nizatidine, and ranitidine,
the muscarinic antagonists, or parasympatholytic agents, is pirenzepine or pipenzolate
the antacids is selected from the group consisting of sodium bicarbonate, aluminium hydroxide, and magnesium hydroxide, and
the proton pump inhibitor is selected from the group consisting of Lansoprazole, Esomeprazole, Rabeprazole, Pantoprazole and Omeprazole
